(19) United States
(12) Patent Application Publication
(10) Pub. No.: US 2018/0082038 A1
(43) Pub. Date: Mar. 22, 2018

(54) SENSOR BASED REORDERING SYSTEM

(71) Applicant: Wal-Mart Stores, Inc., Bentonville, AR (US)

(72) Inventors: Richard M. Blair, II, Bentonville, AR (US); William Ross Allen, Pea Ridge, AR (US); Paul Mayer, Bentonville, AR (US)

(21) Appl. No.: 15/685,060

(22) Filed: Aug. 24, 2017

Related U.S. Application Data
(60) Provisional application No. 62/397,571, filed on Sep. 21, 2016.

(51) Int. Cl. G06F 19/00; G06Q 50/22; G06Q 10/08

(52) U.S. Cl. CPC G06F 19/3456; G06Q 50/22; G06F 19/3462; G06F 19/323; G06Q 10/087

(57) ABSTRACT

Systems, methods and apparatuses for tracking amounts consumable products, generating reminders, and reordering depleted products. The systems include a sensor device comprising a wireless transceiver and an elastic retention member affixing the sensor device to the exterior of the consumable product. The sensor device electronically communicates with a computer system and a product management network. The height of the sensor device on the product container may determine the point of product depletion, signaling the computer system when the amount of consumable product is below the desired level resulting in the performance of a pre-set action. The sensor device calibrates to the density of the product and stores the measurements as sensor data. Alterations to the sensor data are indicative of depletion of the consumable product and initiate the performance of a pre-set action to remind the user to replace the product or automate the replacement of the consumable product.

### CROSS REFERENCE TO RELATED APPLICATIONS

This application claims priority and benefit of U.S. Application No. 62/397,571 entitled SENSOR BASED REORDERING SYSTEM, filed on Sep. 21, 2016, the contents of which are hereby incorporated by reference.

### TECHNICAL FIELD

The present disclosure relates generally to systems, methods and apparatuses for automatically detecting the depletion of a product and reminding, reordering or replacing the depleted products.

### BACKGROUND

Consumable items such as food, beverages, toiletries and cosmetics are typically sold and stored in product containers. Often, inventory of the amount of product remaining in the containers is evaluated to determine whether a replacement or refill of the product being evaluated should be purchased. Identifying the amount of contents remaining in product containers has been traditionally determined manually. For example, consumers periodically perform a visual inspection or weight based evaluation of inventory contents remaining in food, beverage containers, toiletry items and cosmetics to identify a shopping list of items to purchase from a grocery store. Based on the visual inspection or lifting up the product container, the consumer often makes a logical evaluation of whether or not to purchase or schedule a purchase to replace the inspected item. This manual process is laborious, imprecise, error prone and relies on the consumer to remember to perform the inspection of the item before the contents of the item have been depleted. For example, it is often difficult for a person to visually determine an amount of liquid beverage remaining in a bottle with precision in a reliable manner. In commercial settings, the amount of time spent by an employee to manually inspect the amount of inventory remaining also represents a real employment cost to the employer. Therefore, there exists a need for a better way to determine the amount of content remaining in a container and provide a customizable response when the contents of a container reach a minimally acceptable level.

### SUMMARY

A first embodiment of the present disclosure provides a method for reordering a consumable product comprising the steps of: affixing a sensor device comprising a sensor and an adjustable band to an exterior surface of a container confining the consumable product, wherein a height of the sensor device affixed to the container is at a desired level of the consumable product remaining before triggering a pre-set action; calculating, by the sensor device, a density measurement of the consumable product inside the container; detecting, by the sensor device, a change in the density measurement, indicating depletion of the consumable product below the desired level; and transmitting, by the sensor device, a signal to perform the pre-set action as a function of the detecting step.

A second embodiment of the present disclosure provides a computer system comprising a central processing unit (CPU); a memory device coupled to the CPU; a sensor device, having a sensor and an adjustable band, the sensor device coupled to the CPU; and a computer readable storage device coupled to the CPU, wherein the storage device comprises program code executable by the CPU via the memory device to implement a method for reordering a consumable product comprising the steps of: calculating, by the sensor device, a density measurement of the consumable product stored inside a container; detecting, by the sensor device, a change in the density measurement indicative of a removal of the consumable product below a desired level, the desired level being indicated by a position of the sensor device affixed to the container; and transmitting, by the sensor device, a request to perform a pre-set action by the CPU, as a function of the detecting step.

A third embodiment of the present disclosure provides a computer program product comprising: a computer-readable storage device; and a computer-readable program code stored in the computer-readable storage device, the computer-readable program code comprising instructions executed by a central processing unit (CPU) of a computer system implementing a method for reordering a consumable product comprising the steps of: calculating, by the CPU, a density measurement of the consumable product stored inside a container from data collected by a sensor device electronically paired to the computer system; detecting, by the CPU, a change in the density measurement from the data collected by the sensor device, indicating a removal of the consumable product below a desired level, wherein the desired level is indicated by a position of the sensor device affixed to an exterior surface of the container; receiving, by the CPU, a signal to perform the pre-set action from the sensor device; and performing, by the CPU, the pre-set action as a function of the detecting step.

### BRIEF DESCRIPTION OF THE DRAWINGS

FIG. 1 depicts a block diagram of an embodiment of a system for reordering a consumable product.

FIG. 2a illustrates an embodiment of a system for reordering a consumable product.

FIG. 2b illustrates an embodiment of a computer system electronically pairing with a sensor device.

FIG. 3 illustrates an embodiment of a system for reordering a consumable product identifying a consumable product.

FIG. 4 depicts an embodiment of a sensor device affixed to a consumable product at varying detection heights.

FIG. 5a depicts an embodiment of a sensor device detecting an presence of a consumable product.

Sensor 5b depicts an alternative embodiment of a sensor device detecting a presence of a consumable product.

Sensor 5c depicts another alternative embodiment of a sensor device detecting an absence of consumable product.

FIG. 6 illustrates an embodiment of a system of the presence disclosure triggering a pre-set as a function of the geolocation of the system.

FIG. 7 depicts an embodiment of a method for reordering a consumable product.

FIG. 8 depicts a block diagram of a generic computer system for reordering a consumable product.

### DETAILED DESCRIPTION

Although certain embodiments are shown and described in detail, it should be understood that various changes and modifications may be made without departing from the scope of the appended claims. The scope of the present disclosure will in no way be limited to the number of constituting components, the materials thereof, the shapes thereof, the relative arrangement thereof, etc., and are disclosed simply as an example of embodiments of the present disclosure. A more complete understanding of the present embodiments and advantages thereof may be acquired by referring to the following description taken in conjunction with the accompanying drawings, in which like reference numbers indicate like features.

As a preface to the detailed description, it should be noted that, as used in this specification and the appended claims, the singular forms “a”, “an” and “the” include plural referents, unless the context clearly dictates otherwise.

### Overview

Embodiments of present disclosure improve upon systems and methods for manually assessing the contents of a consumable product and whether the contents of the consumable product should be replaced or reordered. Often, user's manually evaluating the content remaining in a container can be imprecise. Embodiments of the present disclosure eliminate guessing and arbitrary evaluation techniques typically performed by individual consumers deciding whether or not to reorder or replace a consumable product. Embodiments of the present disclosure may use sensor based technologies to identify an amount of consumable product remaining within a product's container and upon reaching a pre-determined level of depletion, the sensor device may transmit a signal to one or more computing systems capable of performing a pre-determined action selected by the consumer, including but not limited to ordering a replacement product for delivery, scheduling a time to pick up a replacement product at a store, adding the product to a designated list, automatically queuing the product for pickup upon entering a store or transmitting a reminder to the consumer upon entering a store that may sell a replacement product.

Embodiments of the system, methods and apparatuses offer solutions for identifying a consumer product, tracking the contents of the consumer product and performing an action relating to the consumer product being tracked. Embodiments of the disclosure may include a sensor device comprising a sensor, a power source, a wireless transmitter or transceiver and an elastic retention member. The sensor device may be electronically paired or placed into communication with a computer system. The computer system may identify the sensor device by an identification number, color code, mac address, IP address or other unique identifying characteristic. The computer system may scan a consumable product or a tag placed on the consumable product using a scanner or object identifying camera and once scanned, the computer system may assign the specific product identified to the sensor device.

The consumer may utilize the sensor of the sensor device by affixing the sensor device to an exterior surface of the scanned product at a height selected by the consumer. The height of the sensor device may determine the point of product depletion that the sensor device alerts the computer system that the amount of consumable product is below the desired level. Embodiments of the sensor device may detect the levels of consumable product using one or more sensors that calculate the density of the product within the consumable product container. As a function of the density calculation, the sensor may emit a signal or transmit sensor data to the computer system. Once the consumable product inside the product container is reduced below the sensor's selected height, the density will change due to the lack of product present in the container. The change in the density calculation and/or the sensor data may indicate to the paired computer system that the amount of product has been reduced below the desired level, triggering the computer system to perform one or more pre-set actions selected by the consumer.

### System for Reordering a Consumable Product

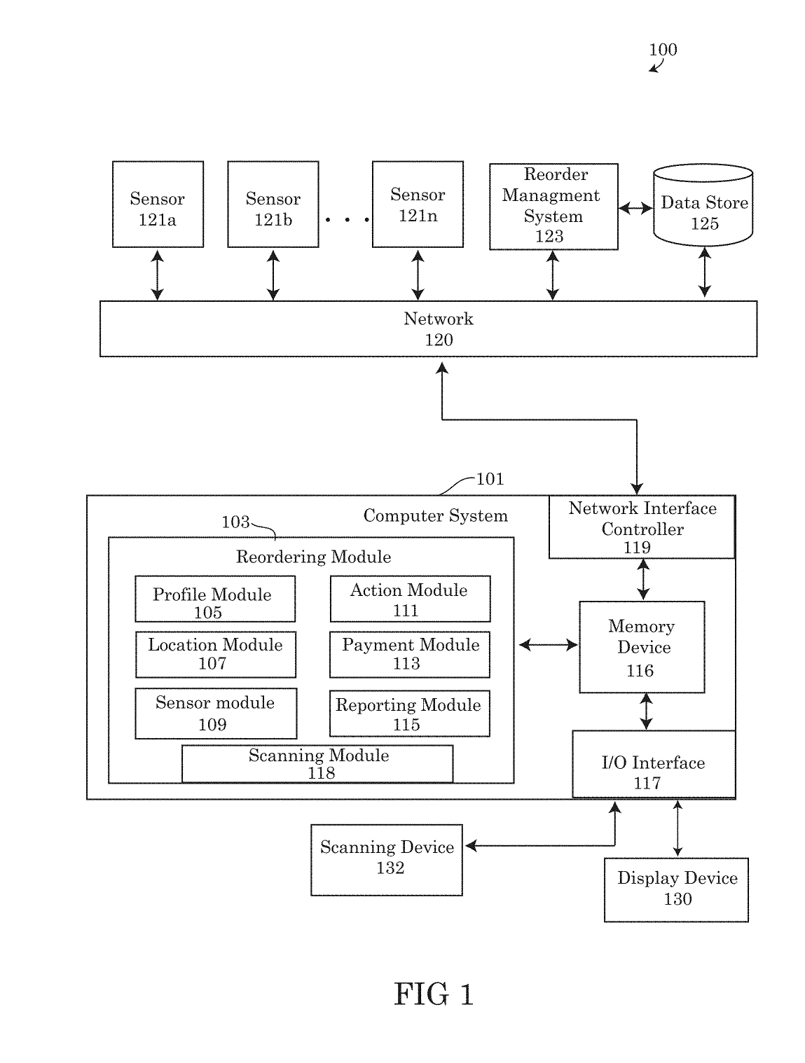

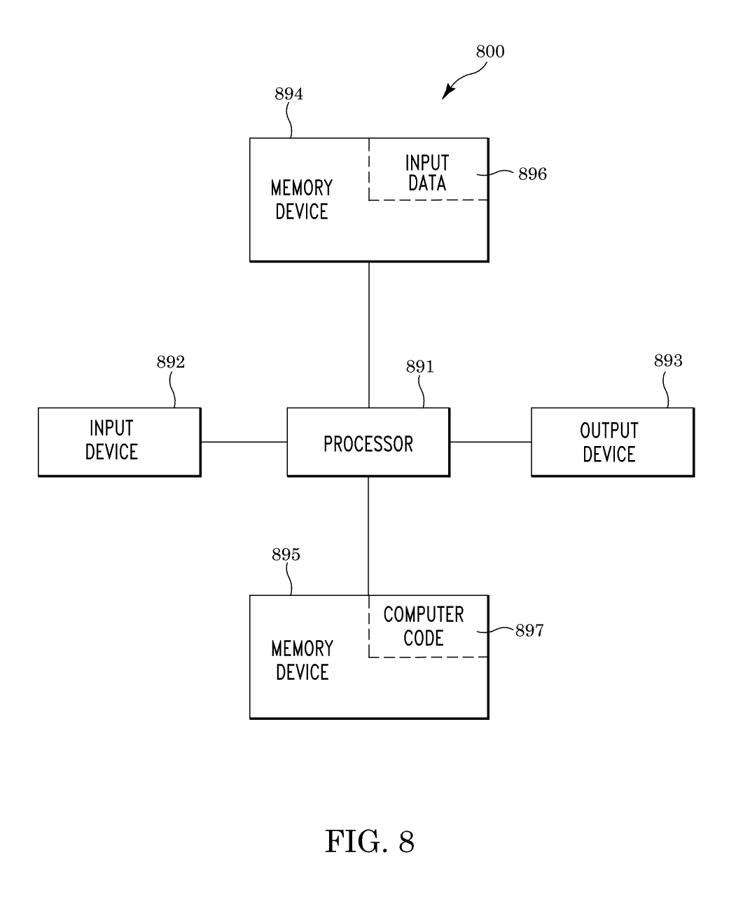

Referring to the drawings, FIG. 1 illustrates a block diagram of a system 100, for reordering a consumable product, consistent with the disclosure of this application. Embodiments of system 100 may comprise a computer system 101. The computer system 101 may be a specialized computer system, having specialized configurations of hardware, software or a combination thereof as depicted in FIGS. 1-6 of the present disclosure in some embodiments. Embodiments of the computer system 101 may further comprise one or more elements of the generic computer system 800 of FIG. 8, described in detail below. The elements of the generic computer system 800 may be integrated into the specialized computer system 101 of FIGS. 1-6.

Embodiments of the computer system 101 of system 100, for implementing a method of reordering a consumable product, may include a reordering module 103. The term “module” may refer to a hardware module, software-based module or a module may be a combination of hardware and software resources of the computer system 101 and/or resources remotely accessible to computer system 101. A module, whether comprising hardware, software or a combination of resources thereof, may be designed to implement or execute one or more particular functions, tasks or routines of the reordering system 100. Embodiments of hardware-based modules may include self-contained components such as chipsets, specialized circuitry and one or more memory devices. A software-based module may be part of a program code or linked to program code containing specific programmed instructions loaded into the memory device 116 of the computer system 101 and/or a remotely accessible memory device (not shown) such as a network accessible computer system, for example a web server. In the system 100, shown in FIG. 1, the network accessible computer system may include the reorder management system 123, which may be acting as a web server or application server. In some embodiments, the reordering module 103, as described in this application, may be partially or entirely incorporated into the reorder management system 123. In such an embodiment, the computer system 101 may remotely access each of the features of the reordering module 103 remotely via network 120.

Embodiments of the reordering module 103 may include one or more sub-modules which may be designated with performing one or more tasks or routines of the reordering module 103. In some embodiments, the reordering module 103 may include a profile module 105, a location module 107, a sensor module 109, a scanning module 118, an action module 111, a payment module 113 and a reporting module 115.

The profile module 105 may perform one or more tasks related to identifying a user profile, linking the user's profile to the system 100 and/or an online accessible account, requesting, receiving and transmitting user information from the computer system 101 to the reorder management system 123 and/or each sensor 121a, 121b . . . 121n of the system 100. A user accessing the computer system 101 of the system 100 may track the user's use of system 100, the consumable products being purchased and reordered by system 100 and the profile module 103 generate custom lists or suggestions of products that may be reordered by the system 100. The profile module 105 may be responsible for saving, storing and retrieving user profiles and configuration settings for each sensor 121a, 121b . . . 121n (hereinafter referred to as sensor 121) managed by the system 100. Embodiments of the profile module 105 may receive input data from the user, which may indicate the presence of one or more particular users and in response to receiving the input data indicating the presence of a particular user, the profile module 105 may select the appropriate user profile and load the customized settings into the memory device 116 of the computer system 101.

Embodiments of the profile module 105 may identify the user of the computer system 101 by verifying the user attempting to access a particular user profile stored by the computer system 101, data store 125, the reorder management system 123 or other network accessible computer device. Verification of a user may include a request by the profile module 105 for the user to produce one or more types of authentication credentials demonstrating a user's rights to access the requested user profile stored by the profile module 105 or reorder management system 123. For example, the user may provide password authentication, smart card authentication or a biometric authentication. For instance, the profile module 105 may receive biometric data for the purposes of authentication and identification of the user through one or more biometric input devices or a scanning device 132. Authenticating devices may use visual or audio devices such as a camera, biometric camera and/or microphone for the purposes of facial recognition, iris scanning, fingerprint scanning, voice recognition, hand recognition, signature recognition or other forms of biometric input known by individuals skilled in the art.

Authenticating devices such as the scanning device 132 may integrated into computer system 101 or may be a separate or stand-alone devices connected to the computer system 101, for example through a USB, PCI, PCIE, Fire wire or other port. The scanning device 132 and other input or output (I/O) devices may be connected to an I/O interface 117 of the computer system 101. An I/O interface 117 may refer to any communication process performed between the computer system 101 and the environment outside of the computer system 101, for example the scanning device 132 and display device 130 or other peripheral devices attached to the computer system, including a mouse and keyboard. In some embodiments of system 101, authentication of the user may occur automatically, whereas in alternative embodiments, authentication of the user to the profile module 105 may occur manually by transmitting user-identifying input to computer system 101 or other device comprising the profile module 105.

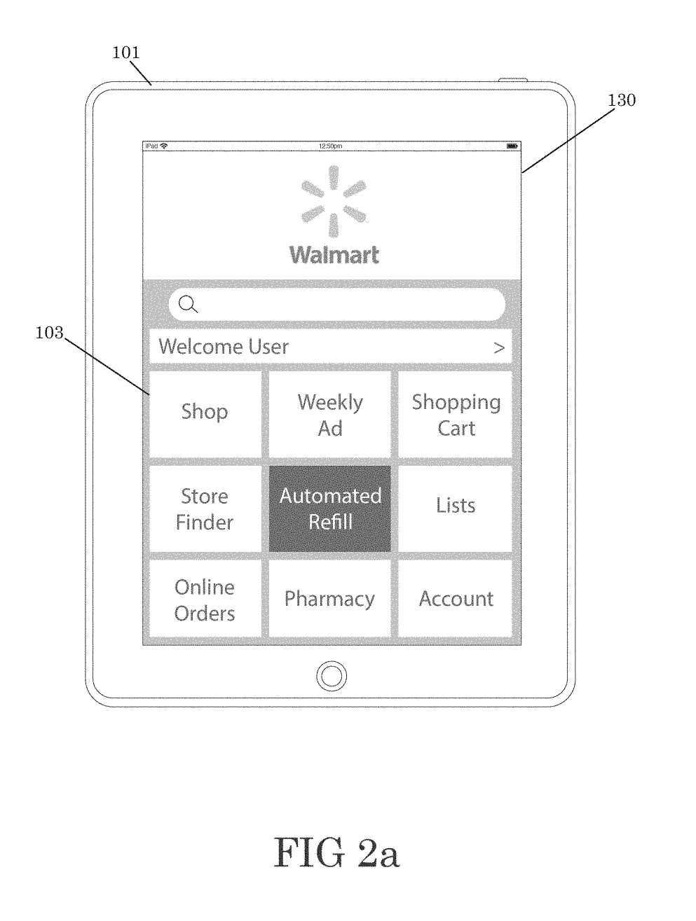

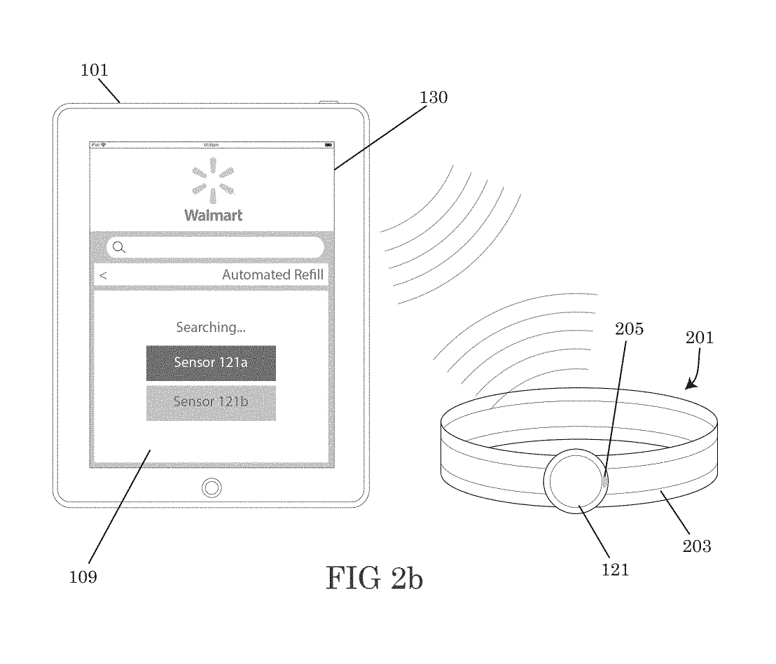

In some embodiments of the reordering system 100, the reordering module 103 may include a location module 107. The location module 107 may include hardware and/or software for identifying the current geographic position or track a series of previous geographic positions of the computer system 101. The location module 107 may be particularly useful when the computer system 101 is a device that is less stationary, such as a mobile communication device, smartphone, tablet or laptop computer. Referring to the drawings, FIG. 2a depicts an embodiment of a mobile computing device operating as computer system 101 loaded with reordering module 103 displayed by a display device 130 of the computer system 101.

In some embodiments, the location module 107 may include hardware and software capable of utilizing the global positioning system (GPS). For example, a GPS antenna, transmitter, transponder, transceiver or receiver may be connected to the computer system 101 or integrated into the hardware of location module 107. The location module 107 may acquire the GPS location of the computer system 101 from GPS satellites and may store location data of the computer system 101 in the memory device 116, the profile module 105 or remotely in the data store 125 of the reorder management system 123.

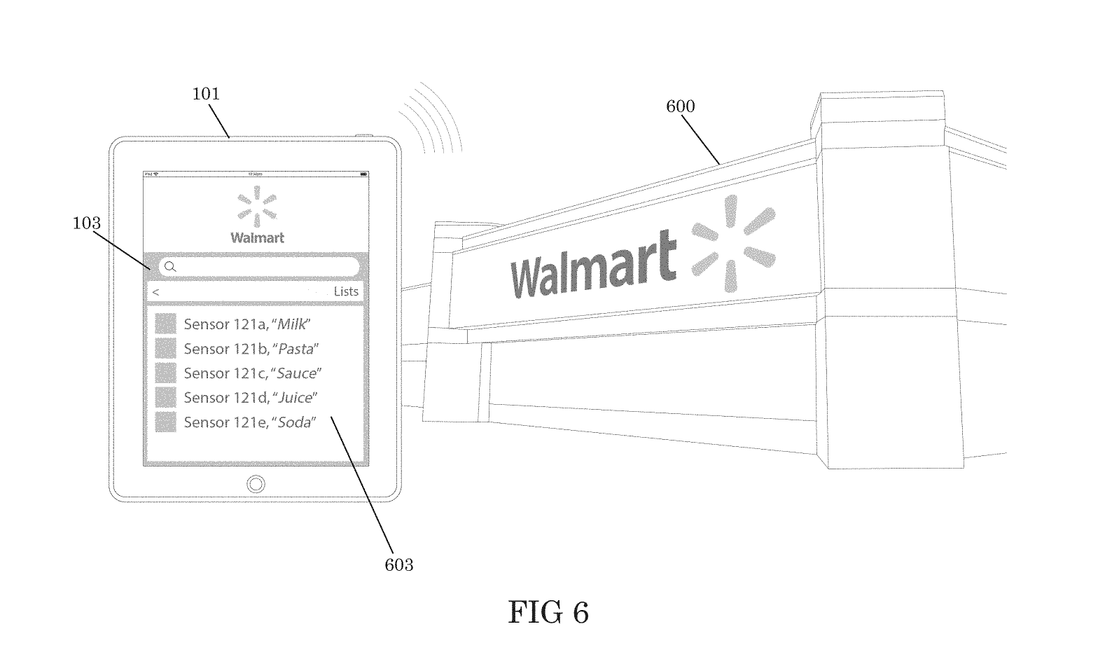

The location module 107 may continuously and/or periodically obtain, save and track the geographical position of the computer system 101. The location module may periodically compare the current location of the computer system 101 with one or more mapping programs to identify whether or not the computer system has entered a specific location. For example, the computer system 101 or reorder management system 123 may use the location data collected by the location module 107 to identify the proximity of the computer system 101 to one or more different stores where a consumable product being tracked may be purchased or reordered by the reordering system 100. As the user comes into proximity with a particular store, the system 100 may schedule a replacement product for pickup, remind the user about retrieving a replacement product at the nearby store, alert the store to have the replacement product ready for the client or transmit an advertisement for the product to the computer system 101 of the reordering system 100. In some embodiments, the location module 107 may establish one or more geofences around a particular location or storefront. When the location module's 107 location data indicates that a user has crossed a geofence barrier, the system 100 may perform one or more pre-set actions described herein, as a function of crossing the geofence barrier. An example of using proximity of the computer device to the storefront 600 can be seen in FIG. 6. The computer system 101 is depicted as being positioned outside of a store and as a function of the proximity to the store, the computer system 101 displays a reminder 603 describing a list of consumable products to purchase.

Embodiments of the reordering system 100 may further include a sensor module 109. The sensor module 109 may perform the task or function of transmitting, receiving, calculating and/or interpreting sensor data collected by one or more sensors 121a, 121b . . . 121n. It should be noted that elements depicted in the figures having reference numbers that include a series of sub-letters followed by ellipses and an n^{th} sub letter, for example the sensors 121a, 121b . . . 121n, may signify that the embodiments comprising the element are not limited only to the amount of elements specifically shown in the drawings, but rather, the ellipses between the letters and the n^{th} element indicate a variable number of similar elements of a similar type that may be present. For instance, with regard to sensors 121, any number of a plurality of sensors 121 may be present, including sensor 121a, sensor 121b and a plurality of additional display sensors up to and including the n^{th} number of sensors 121n, wherein the variable “n” may represent the last element in a sequence of similar elements shown in the drawing.

The sensor module 109 sending and receiving data from each of the sensors 121, may electronically communicate with each of the sensors 121 over a network 120. As shown in FIG. 1, the network 120 may be a group of computer systems or other computing devices linked together through communication channels to facilitate communication and resource sharing among the computer systems and hardware devices of connected to the network. Examples of network 120 may include a local area network (LAN), home area network (HAN), wide area network (WAN), back bone networks (BBN), peer to peer networks (P2P), campus networks, enterprise networks, the Internet, cloud computing networks and any other network known by a person skilled in the art. Embodiments of the computer system 101 may connect to the network 120 via a network adapter or network interface controller 119. The network interface controller 119, as shown in FIG. 1 may allow for data to be transferred and received between the computer system 101 and each of the devices connected to the network 120.

In some embodiments, network interface controller of computer system 101 may be a wireless interface, which may include a wireless transmitter, receiver or transceiver. The wireless signals and data being transmitted between each of the devices connected to network 120 may be transmitted wirelessly between the sensors 121 or other devices of network 120 and the computer system 101. For example, the wireless network may be established using Wi-Fi, Bluetooth, Bluetooth low energy (BLE), ZigBee, WiMAX, light fidelity (Li-Fi), infrared or other wireless communication protocols known by those skilled in the art.

In some embodiments, the sensor module 109 may utilize the computer system 101 to electronically pair the computer system with each of the sensors 121. Each of the sensors 121 may have a unique identification code, IP address, MAC address, associated color or customized naming protocol that may be identifiable to the sensor module 109. The unique identifier of each sensor 121 may allow for the sensor module 109 to track the origins of sensor data being sent and received by a particular sensor 121. An example of pairing the sensor 121 with the computer system 101 is shown in FIG. 2b. As depicted, the sensor module 109 may be able to scan and identify one or more sensors within range to the computer system 101 and electronically communicate with the sensor 121 via wired or wireless methods. The sensor module 109 may allocate specific commands to each sensor 121 individually, identify when a particular sensor may be suffering from a low battery or malfunction and specifically correlate each sensor 121 to a particular consumable product. Moreover, embodiments of the electronically paired sensor may allow for the sensor module 109 to customize sensor settings and pre-set actions for each of the paired sensors 121 identified by the computer system 101.

In some embodiments, the sensor module 109 may transmit identifying information about each of the sensors 121 placed into electronic communication with computer system 101 to the profile module 105 and/or the reorder management system 123. The sensors 121 electronically communicating with computer system 101 may be linked to a specific user profile managed by the profile module 105 of the computer system 101 or stored in a profile database of the data store 125 connected to the reorder management system 123. For example, the profile module 105 may manage an online account, such as a Walmart.com user account, which may be used for purchasing products, receiving discounts and/or tracking the order history of the account user.

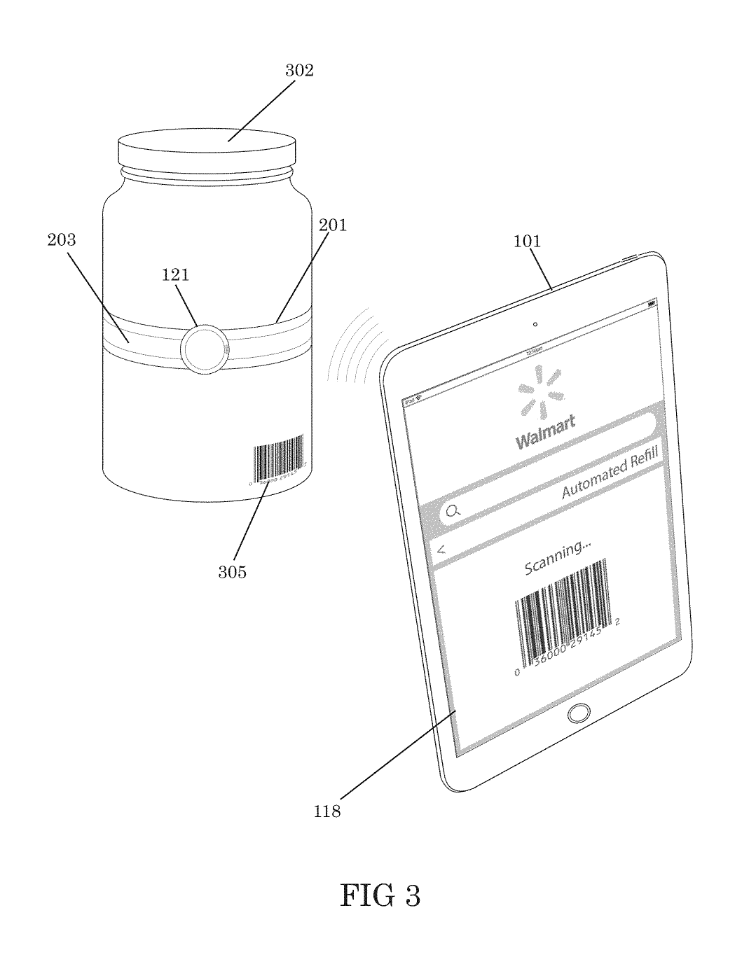

Embodiments of sensors 121 may be integrated into a sensor device 201, such as the sensor device 201 shown in FIG. 2b. Embodiments of the sensor device 201 may include an adjustable sized band 203 or similar attachment device capable of affixing the sensor device 201 to an exterior surface of a consumable product, as shown in FIG. 3, 4, 5a-5c. The adjustable band 203 may be constructed out of any elastic materials that are capable of adjusting or stretching to a desired size. For example, the adjustable band may be constructed out of one or more materials selected from various plastics such as polyvinyl chloride (PVC), natural or synthetic rubbers, vinyl, silicone, nylon, latex and polyester.

Embodiments of the sensor device 201 may include a power source. The power source may be any known power source capable of providing enough energy to enable each of the components of the sensor device 201. The power source may be a rechargeable or non-rechargeable energy source. Embodiments of the power source may be battery powered, solar powered or even light powered. Examples of a battery powered source (either rechargeable or non-rechargeable) may include but may not be limited to alkaline batteries, aluminum-ion batteries, lithium batteries, lithium-ion batteries, magnesium or magnesium-ion batteries, silver-oxide batteries, zinc-batteries, lead-acid batteries, nickel-cadmium batteries and combinations thereof. In some embodiments of the sensor device 201, the sensor device 201 may be rechargeable via a universal serial bus (USB). The sensor device 201 may comprise a USB port 205 capable of receiving a USB, mini-USB or micro-USB cable capable of transmitting energy from a computer system 101 wall outlet or other electrical energy source to the power source of the sensor device 201 in order to recharge the power source. In some embodiments of system 100, the sensor device may transmit a notification or alert to the computer system 101 using a wired or wireless transmitter or transceiver, when the power source of the sensor device 201 has become depleted or is lower than a pre-determined level. The computer system 101 may display the alert on a display device 130 connected to the computer system in order to alert the user that it is time to replace or recharge the power source providing energy to the sensor device 201.

Embodiments of the wireless transmitter or transceiver may transmit data collected by the sensor device 201, as well as alerts to the computer system 101. The wireless receiver or transceiver may also receive instructions, commands or signals transmitted by the computer system 101. The wireless transmitter, receiver or transceiver may be any type known by a person skilled in the art for connecting an electronic device to a computer network 120 or electronically pairing electronic devices to a computer system 101. For example, in some embodiments, the sensor device may be equipped with a Wi-Fi, Bluetooth, Bluetooth low energy (BLE), ZigBee, WiMAX or light fidelity (Li-Fi) antenna. The sensor device 201 may wirelessly receive activation instructions from the computer system 101 to begin collecting sensor data and density measurements of consumable products in some embodiments. In alternative embodiments, the sensor device 201 may include a calibration or activation button. The activation or calibration button may be physically pressed by the user to activate the sensor device 201

Upon activating the sensor device 201, the sensors 121 of the sensor device 201 may emit a signal to measure the density of the attached consumable product. The signal may be constantly emitted or intermittently emitted at a rate set by the sensor device 201 or the computer system 101. For example, the sensor 121 may emit a signal every 5, 10, 15, 20, 30, 45, 60 minutes or more, depending on the amount of time set. In some embodiments, the sensor device 201 may include multiple sets of sensors. For example, the sensor device 201 may include a sensor for calculating the density of the consumable product and a separate set of sensors for identifying movement or motion of the sensor device 201. The second set of sensors may identify movements such as a change in orientation, which may be indicative of a pouring motion of a consumable product.

In some embodiments, the sensors 121 may not only measure the density of the consumable product at a set interval, the sensors 121 may also emit a measuring signal after the second set of sensors have been triggered (i.e. after product has been poured from the container). In some embodiments, the measuring signals emitted by the sensor 121 may be emitted for an extended period of time. For example 1-10 mins, 10-30 mins, 30-60 mins, etc. By emitting a signal for measuring the density over an extended period of time, the sensor 121 may avoid falsely identifying the consumable product as being below the desired level of product before commencing a pre-set action.

In the exemplary embodiment of the sensor 121, the sensor 121 may be a capacitance sensor. The capacitance sensor may calculate the changes in density of the consumable product by using a dielectric constant to determine the differential in capacitance between densities both in the presence of the consumable product and the absence of consumable product. Initially, upon calibrating the sensor 121 to a container comprising consumable product, an initial capacitance may be calculated wherein the capacitance is measured in the presence of the consumable product within the container. Upon the removable of the consumable product from the container below the height the sensor is set at, the capacitance value changes (due to the absence of product), therefore signaling to the sensor 121 and computer system 101 that the consumable product is no longer present in the container at a height equal to or greater than the height of the sensor 121.

In some embodiments of system 100, the sensor device 201 may identify the changes in the sensor 121 signal and in response to the changes in the measuring signal, transmit a request to the computer system 101 to proceed with performing the pre-set action designated for the particular sensor 121 experiencing a change in capacitance. In an alternative embodiment, the change in density or output signal of the sensor may be calculated by the computer system 101 receiving sensor data. In such an embodiment, the computer system 101 may be determining as a function of the received sensor data whether or not the pre-set action should be triggered.

In some embodiments, the sensor 121 may include one or more classes of sensors including but not limited to an optical sensor, light sensor, electro-optical sensor, photoelectric sensor, optical switch, proximity sensor, motion sensor, acoustic sensor, mechanical sensor environmental sensor, magnetic sensor or any other type of sensor that may detect the presence or absence of a consumable product within a container by placing the sensor 121 on the exterior surface of the consumable product's container. Embodiments of optical sensors, electro-optical sensors and optical switches integrated into the sensor device 201 may convert light rays into electric signals, thereby allowing for the measurement of a physical quantity of light than can be translated into a form that is readable by the sensor device 201 or a computer system 101 receiving the sensor data. Embodiments of the optical sensors, electro-optical sensors and optical switches may include a trigger that may react to a change or alteration to the signal being emitted by the optical or electro-optical sensor. When a measurable change occurs to the light ray being emitted, the optical or electro-optical sensor may operate as a photoelectric trigger and either increase or decrease an electrical output which may be sent to a separate instrument (i.e. computer system 101) translating or interpreting the sensor data received from the sensors 121.

Embodiments of the present disclosure utilizing optical, light, photoelectric or electro-optical sensors may be particularly useful for consumable products that may have a container with a transparent, translucent or slightly opaque container that allows light to pass through. The presence of the consumable product may affect the distance that the light may travel and thus when the product is removed, the calculation of the light passing through the container may be altered thus indicating to the sensor 121 and/or computer system 101, a lack of consumable product inside the container at the user-selected height of the sensor.

In alternative embodiments, the sensor 121 may be a radar sensor. The radar sensor may use micro-power impulse radar, similar to certain types of modern stud finders. The radar sensor may emit low-power ultra-wideband radar to sense and measure the distance of objects in proximity to one another. Initially, when the container holder the consumable product is at a height greater than or equal to the height of the sensor 121, detects the proximity of the consumable product below the exterior surface of the container's packaging. Likewise, when the consumable product is below the sensor's level on the exterior surface of the container, the signal received by the radar's return wave differs due to the emitted signal travelling further in the absence of consumable product.

Alternatively, an acoustic sensor may also be integrated as part of the sensor device 201. The acoustic sensor may rely on the modulation of acoustic waves to sense physical whether the consumable product is detectable at the height of the sensor 121 positioned on the container of the consumable product. Embodiments of the acoustic sensor may transduce an electrical signal input into a mechanical wave and then may transduce the wave back into an electrical signal, whereby changes in amplitude, frequency and time-delay between the input and output electrical signals can be used to measure the presence or absence of the consumable product.

In some embodiments of the sensor device 201, the sensor 121 may be a range-finding sensor. Similar to the acoustic sensor described above, the range finding sensor may detect the presence of obstacles in the path of the sensor by propagating sounds waves. These sound waves may bounce off of a reflective surface and return to the sensor. The sensor device 201, or the computer system 101 connected to the sensor device 103 may calculate the amount of time it takes for the wave to return to the range finder sensor. Based on changes in the return time, the sensor device 201 or the computer system 101 may conclude the presence or absence of consumable product within the container holding the consumable product.

Alternative embodiments of the sensor device 201 may comprise a proximity sensor. A proximity sensor may operate by emitting an electromagnetic field or a beam of electromagnetic radiation (such as infrared). The proximity sensor may identify changes in the field or a return signal. Different of proximity sensors may be used depending on the type of packing or consumable product intended to be identified. For example, the proximity sensor may be a capacitive, photoelectric or inductive proximity sensor.

Each of the sensors 121 electronically communicating with the computer system 101 may be assigned a specific type of consumable product to track and reorder. In some embodiments, the name or type of product being tracked by the sensor may be manually inputted or selected by the user from a database of consumable products. In the exemplary embodiment, the system 100 may scan the UPC 305, QR code, bar code or utilize an object recognition camera to identify the consumable product associated with each of the sensors 121. In some embodiments, the reordering module 103 may include a scanning module 118. The scanning module may perform the tasks of scanning and identifying the consumable product as well as transmitting information about the consumable product to the profile module 105, sensors 121, the reorder management system 123 and/or the data store 125.

The scanning module may collect consumer product information and identify the consumer product using one or more scanning devices 132, which may be connected to computer system 101. The scanning device 132 may include a camera or barcode reader (or scanner). In some embodiments, the camera may utilize barcode reading software or object recognition software loaded in the memory device 116 of the computer system. For example, in the exemplary embodiment, the camera may be an integrated camera of a mobile communication device having bar code, QR code, UPC 305 or object recognition applications loaded in the memory of the device. By focusing the camera or other scanning device 132 on the consumable product, label or bar code, the scanning device may identify the type of product that may be eventually reordered as well as the size of the product and the product's composition (ingredients). In some embodiments the computer system 101 may save location information where a replacement of the scanned product may be purchased or replaced and the stored information may be queried by the system 100 at a later point in time.

FIG. 3 provides an example of computer system 101 scanning the consumable product 302 using the scanning module 118. In this particular embodiment, the computer system 101 may use the frontward facing camera of the computer system to focus on the UPC code 305 printed on the packaging of the consumable product 302. As shown in the drawing, the scanning module registers the UPC 305 or other unique identifier being scanned. The scanning module 118 may then compare the scanned code with a database of product codes to identify the consumable product 302 that was scanned.

Embodiments of the reordering module 103 may further comprise an action module 111. The action module 111 may receive input data from the user, indicating one or more different types of pre-set actions that may occur upon a sensor 121 identifying that a consumable product has depleted to a level below the position of the sensor 121 currently affixed to the exterior surface of the consumable product's container. In some embodiments, the user may select or pre-set actions that may occur as a function of the consumable product being removed from the container. Once the requisite amount of consumable product has been removed from the container to below the position of the sensor 121, the action module 111 may implement the pre-selected action.

The implementation of the pre-selected action by the action module 111 may vary, depending on the particular action selected. The types of actions that may be performed by the action module 111 may include scheduling a delivery of a consumable product, scheduling a pickup of the consumable product, adding the consumable product a virtual list maintained by the computer system 101 or a remotely accessible computer system, displaying an alert indicating the user of the depletion of the consumable product, queuing the consumable product for purchase at a local store as a function of the computer system's 101 geolocation or displaying a reminder to the user as a function of the computer system's geolocation. Embodiments of the action module 111 may independent the same or different actions for each of the independent sensor devices 201.

In an embodiment of the action module 111, wherein the user has selected the pre-set action of scheduling a delivery, the action module may, upon receiving data from the sensor module 109 indicating the depletion of the consumable product, send a service request (for example an API call) via network 120 to the reorder management system 123. In some embodiments the service request may include information describing the consumable product being replaced. In alternative embodiments, the product information of the consumable product associated with the particular sensor reporting the depleted consumable product, may have been previously stored by the reorder management system 123 when the product was scanned and linked to the sensor 121 and/or linked to the user's account.

Upon receiving the service request, the reorder management system 123, which in some embodiments may be a web server or application server, may fulfill the service request transmitted by computer system 101. The reorder management system may schedule the delivery of the requested consumable product, track the product order, correlate the order to the user's profile and account maintained by the reorder management system as well as transmit a confirmation notification to the computer system confirming the details of the delivery request.

In an alternative embodiment, wherein the user has selected a pre-set action of the action module 111 to schedule a pickup, the action module 111 may transmit a service request via computer system 111 to the reorder management system 123. The service request may include not only the user profile data and consumable product data but may also include location data provided by the location module 107. Upon receiving the pickup request, the reorder management system 123 may query the locations of available stores within a specified radius of the location data provided and transmit to the computer system 101 a plurality of pickup options. The pickup options may include the store location, a date and time when the consumable product may be ready for pickup. The user may subsequently select the store and pickup date desired from the presented options. Alternatively, the user may pre-select a store location or request a favored store location where the pickup may be placed with. Based on the user's location, date and time selection, the reorder management system 123 may transmit the pickup request and profile information to the selected store in order to arrange the pickup as agreed upon by the user.

In some embodiments, where the pre-set action may include adding the consumable product to a virtual list, the virtual list may be maintained by the computer system 101, a network accessible computer system such as reorder management system 123 or a remotely accessible data store 125. Upon receiving the sensor data from the sensor module 109, the action module 111 may load the virtual list in the memory device 116, if the virtual list is stored locally on the computer system 101. Likewise, if the virtual list is stored by a remotely accessible computer system, the action module 111 may transmit a service request to retrieve the virtual list from the network resource and then upon receiving the virtual list data, load the virtual list in the memory device 116 of the computer system. The action module 111 may edit the virtual list by adding the consumable product to the list automatically for the user. Thus, when the user opens the virtual list in the computer system 101 (or any other computer system), the virtual list may display on the display device 130, the consumable product along with any other items that may have been added automatically by the system 100 or that the user may have manually added. In alternative embodiments, instead of retrieving the virtual list from the remotely accessible computer system, the service request transmitted by computer system 101 may include within the request, instructions for the remotely accessible computer to amend the virtual list to include the consumable product.

In an embodiment of the system 100 wherein the pre-set action is selected to display an alert to the user when the consumable product has depleted below the selected level, the action module may, upon receiving sensor data from the sensor module 109 indicating the depletion of the consumable product, instruct the reporting module 115 to display an alert on the computer system 101. For example, the reporting module 115 may display a text or graphical based notification on the display device 130 connected to the computer system or other output device. The notification or alert displayed by the computer system 101 may be a reminder to reorder the consumable product or simply a notification that the particular consumable product's level has dipped below the pre-set height of the sensor device 201 positioned on the exterior surface of the consumable product container.

Similarly, in an embodiment of system 100 wherein the pre-set action is selected to display an alert or reminder as a function of geolocation, the action module 111 may delay the transmission or display of the alert or reminder. The computer system 111 may create one or more geofences around nearby stores that may stock the consumable product that is currently depleted. The action module 111 may transmit a signal to the reporting module 115 to display the alert or notification to the user regarding the depleted consumable product, but only when the location module reports to the reporting module 115 location data that may be within the established geofence. For example, once the user and/or computer system 101 crosses the geofence as determined by the location data retrieved from the location module 107, compared with the location data of the established geofence, the reporting module may transmit and display the alert or reminder to a display device 130. Alternatively, instead of a geofence, the computer system 101 may store GPS location information of local stores or remotely receive GPS location information from the reorder management system 123. The computer system 101 may periodically or continuously track the location data of the location module 107 with the nearest GPS location information of local stores. When the computer system 101 is within a particular distance from the local store (determined as a function of comparing the location information) the reporting module 115 may transmit or display the alert or notification to the user.

In an alternative embodiment of system 100, the user may pre-select the action of the action module 111 to queue the product for pickup at a local store as a function of geolocation. The pre-set action to queue the product for pickup may operate in a manner that may be a combination of the pickup action and the geolocation based alert. Once the action module 111 has received sensor data from the sensor module indicating to the action module to implement the pre-set action, the action module 111 may compare location data received from the location module 107 with location information of local stores stocking the consumable product that has been depleted. When the location data of computer system 101 is either within a specific distance of the local store, or the computer system 101 has crossed the boundary of a geofence surrounding the store, the action module 111 may schedule a pickup of the consumable product at the local store.

In some embodiments, upon meeting the location requirements, the action module 111 may transmit a service request to the reorder management system 123 to handle the pickup transaction. The service request to the reorder management system 123 may include identifying information about one or more consumable products that are being ordered, profile and/or account information of the user as well as location information of the user. Upon receiving the service request, the reorder management system 123 may estimate the time to schedule the pickup of the consumable product(s) and further request the local store to prepare and collect the items for pickup by the account holder at the estimated time. The user, upon entering the nearby local store, may proceed to pick up the consumable product(s) without personally having to collect the products from the local store's shelves. Instead, the consumable product may be pre-arranged and ready for checkout. The computer system 101 may allow for the user to utilize the user's account linked to the reorder management system to pre-pay for the consumable products, or the user may arrange for payment to be processed at the local store upon pickup.

Embodiments of system 100 may include a payment module 113. The payment module 113 may facilitate, negotiate, process and/or complete financial transactions for consumable products reordered or replaced using system 100. The payment module 113 may store payment information tied to a particular user profiled managed by the profile module 105 and/or provide authorization for payments to be processed and accredited to a user's account linked to the reorder management system 123. For example, the action module 111 may select a consumable product for delivery, request the reorder management system 123 and arrange the delivery to the user's address. Subsequently, the reorder management system request permission to charge the product being delivered to the account on file with the reorder management system 123. The payment module 113 may allow the reorder management system 123 to process the payment using the stored payment information of the account or provide new payment information that may or may not be stored by the refill management system.

After payment has been successfully processed by the payment module 113, the payment module may transmit the successful payment information to the reporting module 115. The reporting module 115 may subsequently alert or display the results of the financial transaction between the payment module 113 and the reorder management system 123. The reporting module 115 may transmit and display in some embodiments, a copy of a receipt for the transaction for the reordering the consumable product.

### Method for Reordering a Consumable Product

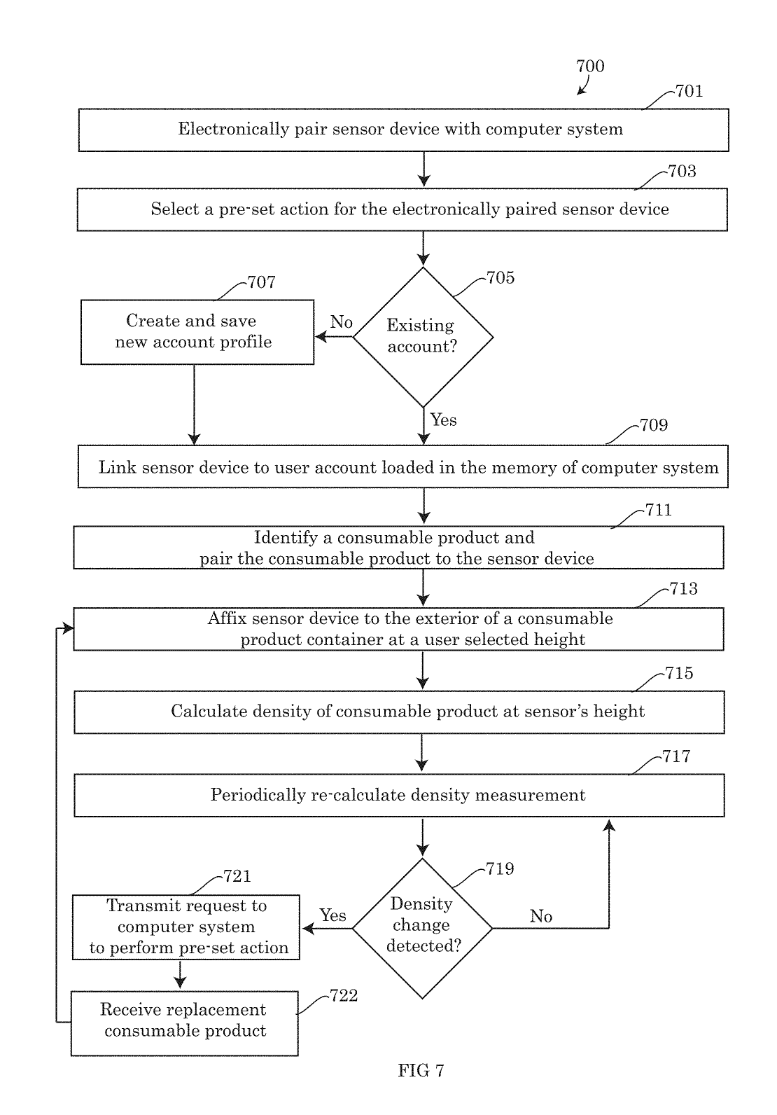

The drawing of FIG. 7 represents an embodiment 700 of a method or algorithm that may be implemented for reordering a consumable product in accordance with the systems described in FIG. 1-6 using one or more computers defined generically in FIG. 8 below, and more specifically by the specific embodiments depicted in FIG. 1-6. A person skilled in the art should recognize that the steps of the algorithm described in FIG. 7 may not require all of the steps disclosed herein to be performed, not does the algorithm of FIG. 7 necessarily require that all the steps be performed in the particular order presented. Variations of the method steps presented in FIG. 7 may be performed, wherein one or more steps may be performed in a different order than presented by FIG. 7.

The method of FIG. 7 may initiate in step 701, by electronically pairing sensor device 201 comprising sensor 121 with computer system 101. The step of electronically pairing or placing the sensor device 201 and the computer system 101 into electronic communication with one another may occur using a communication protocol such as Wi-Fi, Bluetooth, BLE, ZigBee, WiMAX, Li-Fi or any other wireless communication protocol capable of pairing a master and slave devices together. In the exemplary embodiment, the computer system 101 may be the master device and each of the sensors 121 paired to the computer system 101 may be considered the slave devices. Once the sensor 121 and the computer system 101 are placed into electronic communication with one another, the sensor 121 and computer system 101 may transmit and receive data back and forth from one another, including sensor data, activation commands and calibration commands. Moreover, the electronic pairing between the computer system 101 and the sensor 121 may become visible to the computer system 101 with a unique identifier specifically identifying the paired sensor device 201.

In step 703, the computer system 101 may change the settings of the electronically paired sensor device 201. For example, the computer system may select a pre-set action for the computer system 101 to perform as a function of the sensor device's 201 sensor data. The types of pre-set actions that may be selected may include scheduling a delivery of the consumable product, scheduling a pickup of the consumable product, notifying the user of a need to reorder a consumable product, queuing an order of the consumable product upon entering a specified geolocation and transmitting a reminder notification to purchase the consumable product upon entering the specified geolocation. Furthermore, the computer system 101 may also select calibration setting such as the time frame for calculating the density of the consumable product, duration and interval of time the sensor 121 emits signals used to measure density or the presence of the consumable product.

In step 705 of method 700, the system 100 may determine whether or not there is an existing account or user profile to link each sensor device to a user account or profile maintained by the profile module 105. If, it is determined by the system 100 that a user profile or account does not exist or authorized for use by the user, the method may proceed to step 707, wherein the user may create and save a new account and/or user profile. The account or profile may be stored locally in the memory device 116 of computer 101 in some embodiments, while in alternative embodiments, the account or user profile may be stored on a remotely accessible computer device such as a networked computer system, including network accessible web servers or application servers. In the exemplary embodiment of FIG. 1, the user profile or account may be stored and/or managed by the profile module 105, the reorder management system 123, the network accessible data store 125 or a combination of systems thereof. The user profile or account may be created, saved and accessed by the computer system 101 locally or by accessing the user profile account remotely over network 120. For example, by accessing a cloud storage system maintained or web portal.

In step 709 of the method 700, the computer system 101 may link each sensor device 201 to the user profile and/or user account loaded in the memory device 116 of computer system 101 or a remotely accessible user account loaded in the memory device of a network accessible computer system. For example, in step 709, a user logged into computer system 101 may load the user's profile by selecting the profile of the current user from the profile module 105 or by remotely accessing the account via a portal over the network 120. To access the profile, the user may provide any form of authentication described above, including biometric authentication, a user name/passcode identification or smart card identification (such as scanning a key card). Once the user is able to access the user's profile or account information stored by the computer system 101 or a remotely accessible computer system, the user may associate one or more sensor devices 201 to the user's profile or account. By linking a particular sensor 121 to a particular account, the system 100 may track the consumable products being ordered/reordered by the user, the user's preferred locations, preferred stores for receiving consumable products, delivery information (including address) and payment information which may automate the reordering process.

After linking the sensors 121 to the user's account or profile, the method 700 may proceed in step 711 to identify and associate each consumable product with a particular sensor device 201 that may be monitoring the contents of the consumable product. The user may pair a particular type of consumable product to a specific sensor device 201 by selecting the sensor device 201 to be paired with the product, followed by scanning the UPC 305, QR code or other type of barcode containing descriptive information about the consumable product, including the product name, brand, size, etc. In some embodiments, the product may be scanned using an object recognition camera capable of identifying the consumable product from the brand label, shape of the container or other discernable features. The scanning of the consumable product may be performed by aiming a scanning device 132 at the appropriate product's barcode or exterior features and retrieving the data associated with the product being scanned. The data that is read may be saved to the memory device 116, transmitted and stored by the reorder management system 123 or a network accessible data store 125.

In some embodiments, upon identifying the product with the scanning device 132, the computer system 101 may automatically query a database comprising known product information of consumable products. For example, the database of consumable product may be part of the data store 125, or a database stored locally in the memory device 116 of the computer system 101 or reorder management system 123. The computer system 101 may load the product information into the memory of the computer system 101 or reorder management system 123 from the product database. In alternative embodiments, the querying of the database may identify and display product information of a plurality of possible consumable products. The user may manually select the appropriate consumable product that matches the scanned product. In yet another embodiment, the user may manually enter, save and update consumable product information into the product database.

Once the sensor device 201 has been paired with a consumable product chosen or scanned by the system 100, in step 713, the user may affix the sensor device 201 to the exterior surface of the consumable product. The step of affixing the sensor device 201 may be performed by stretching or elongating the elastic material 203 of the sensor device 201 to a size that is greater than the size of the consumable product's container. Once the sensor device 201 has been placed around the packaging of the consumable product, the elastic material 203 of the sensor device 201 may be contracted to the size of the consumable product container, providing a snug hold around the container.

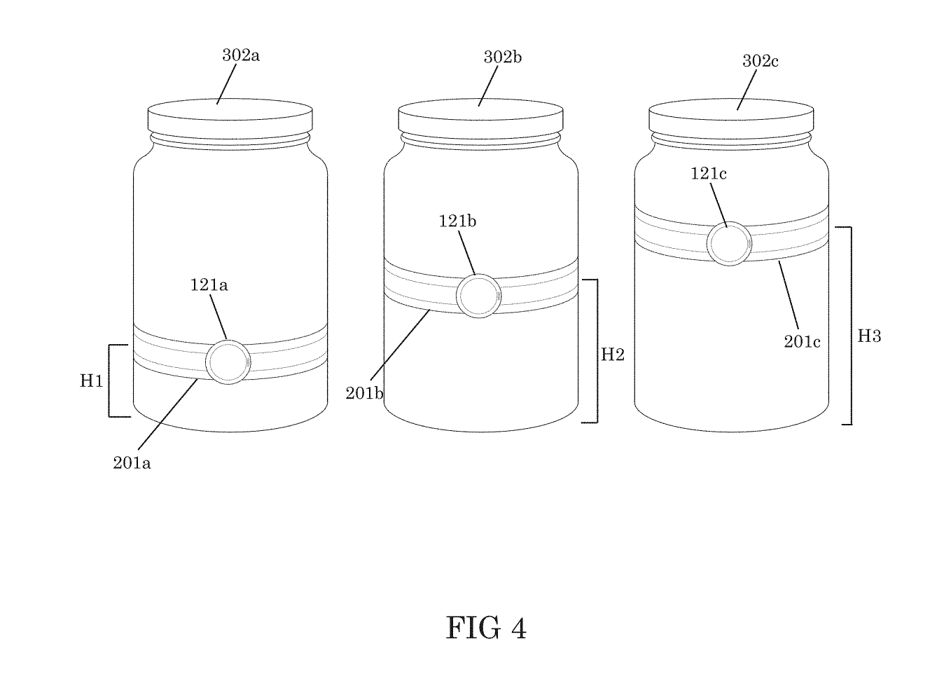

As shown in FIG. 4, the height of the sensor device 201 may be adjusted in relation to the consumable product container. The placement of the sensor device 201 may affect the amount of consumable product remaining before the pre-set action is triggered. For instance, if the sensor device 201a is placed at ¼ of the height (H1) of the product container 302a, then when there is approximately less than ¼ of the consumable product 302a remaining in consumer product container, the pre-set action may be triggered. Likewise, if the sensor device, depicted as sensor device 201b in FIG. 4, is placed approximately at a height (H2) of ½ of the consumer product container 302b, the pre-set action may be triggered once the consumable product remaining in the container is less than the halfway mark where the sensor device 201b resides. Furthermore, increasing the height of the sensor 121c in relation to the container 302c to a height (H3) that is approximately ⅔ of total height of container 302c, then once the consumable product inside the container falls below the height (H3) set by the sensor device 201c, the sensor device 203c may trigger a pre-set action.

In step 715, the sensor 121 of the sensor device 201 may calculate the density of the consumable product at the height the sensor device is placed on the consumable product container. The first density calculation may be considered an initial density calculation. In some embodiments, the initial density calculation may be performed by pressing a calibration button on the sensor device 201 or a transmitted command to the sensor device may be used to instruct the sensor device 201 to perform the initial density calculation. The signal emitted from the sensor 121 may be emitted continuously or intermittently from the sensor device 201 for a length of time designated by the user or the sensor device's 201 default setting. For example, the sample measurement establishing the initial density calculation may be performed by emitting a signal for 30 minutes. In the exemplary embodiment, the signal emitted may be a dielectric constant capable of determining the capacitance of the consumable product as a function of the consumable product's density. A change in the density (caused by a lack of consumable product) may lead to change in the measured capacitance.

Likewise, in alternative embodiments where the sensor is an optical sensor emitting a beam of light or a radar sensor emitted radio waves, the light or radio waves may be emitted during step 715 to calculate the density or identify the presence of consumable product within the container. The absence of consumable product behind the exterior surface of the product's container might affect the path of travel for the light or radio waves emitted or the length of time for the wave to return to the sensor. Thus a change in the presence or absence of the consumer product could trigger the sensor device 201 or computer system 101 to trigger a pre-set action

In step 717, the sensor device 201 may continuously or intermittently re-calculate the density or the initial calculation of the signal emitted during step 715. The frequency of the recalculation and the length of time that step 717 is performed may vary based on the setting of the sensor device 201. The advantage of shorter frequency calculation may be that the consumable product is monitored for depletion more readily, allowing for a faster triggering of the pre-set action as close to the selected level of depletion. Similarly, it may be advantageous to increase the duration of the calculation step in order to prevent an inaccurate determination that the consumable product has been depleted. For example, if the recalculation step 717 initiates while the product is being poured from the container rather than the container being in a stationary position. When the container is inverted, the consumable product may rush toward the top of the product container and leave a void space at the sensor's level. A short recalculation period may result in the sensor 121 or the computer system 101 inadvertently concluding that the consumable product has been depleted. Whereas a longer recalculation period may continue to recalculate for the duration of the inversion of the container and when the container is placed upright again.

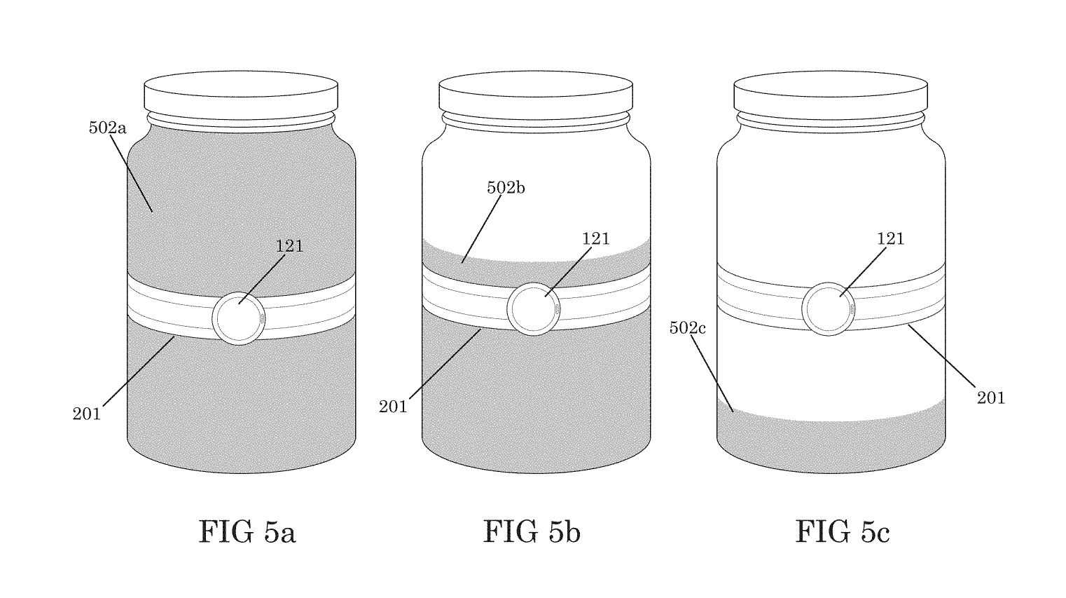

FIGS. 5a-5c may demonstrate a series of points in time wherein the density of the consumable product 502a, 502b, 502c is calculated. At the first interval of time depicted in FIG. 5a, the amount of consumable product 502a is above the sensor 121. As the sensor 121 performs a density recalculation in step 717, the densities of the consumable product 502a and at the initial density calculation in step 715, should be the same due to the presence of the consumable product at a height greater than the sensor 121 in both instances. Therefore, there may be no change in the sensor data. Likewise, at the interval of FIG. 5b, although the consumable product 502b has depleted, the level of depletion has not reached a level below sensor 121. Therefore, the sensor device 201 would not detect a change in sensor data. However, as shown in FIG. 5c, the amount of consumable product 502c remaining at the time samples in FIG. 5c is clearly below the sensor 121. Accordingly, when the recalculation of the density is taken, the absence of the consumable product at FIG. 5c would be viewable as a change in the sensor data and thus trigger a pre-set action to occur.

In step 719, the sensor device 201 or the computer system 101 collecting and receiving the sensor data may compare the measurements taken at step 717 with the initial measurement taken in step 715. If a change in the measurement of step 715 is not detected by the sensors 121 or the computer system 101, the sensor device may proceed with re-measuring the density or presence of the consumable product at the next scheduled interval of time. Conversely, if a density change is detected, the sensor device 201 or the computer system 101 may proceed to step 721.

As a result of the determination of the depletion of the consumable product in step 719, the computer system 101 may, in step 721, perform the pre-set action or receive a request to perform the preset action transmitted from the sensor device. The implementation of the pre-selected action by system 100 may vary depending on the particular action selected by the user in step 703. The types of actions that may be performed in step 721 may include scheduling a delivery of a consumable product, scheduling a pickup of the consumable product, adding the consumable product a virtual list maintained by the computer system 101 or a remotely accessible computer system, displaying an alert indicating the user of the depletion of the consumable product, queuing the consumable product for purchase at a local store as a function of the computer system's 101 geolocation or displaying a reminder to the user as a function of the computer system's geolocation.

If in step 703, the user has selected the pre-set action of scheduling a delivery, the computer system 101 in step 721 upon receiving data from the sensor module 109 indicating the depletion of the consumable product may send a service request (for example an API call) via network 120 to the reorder management system 123. In some embodiments the service request may include information describing the consumable product being replaced. In alternative embodiments, the product information of the consumable product associated with the particular sensor reporting the depleted consumable product, may have been previously stored by the reorder management system 123 when the product was scanned, linked to the sensor 121 and/or linked to the user's account in step 713.

Upon receiving the service request, the reorder management system 123 may fulfill the service request transmitted by computer system 101. The reorder management system 123 may schedule the delivery of the a replacement consumable product similar to the product scanned in step 711, track the product order, correlate the order to the user's profile and account maintained by the reorder management system as well as transmit a confirmation notification to the computer system confirming the details of the delivery request.

In an alternative embodiment, wherein the user has selected a pre-set action in step 703 to schedule a pickup, the computer system 101 may transmit a service request to the reorder management system 123. The service request may include not only the user profile data linked to the sensor device in step 709 and consumable product data identified in step 711 but the request may further include location data provided by the location module 107. Upon receiving the pickup request, the reorder management system 123 may query the locations of available stores within a specified radius of the location data. The options for picking up the replacement consumable product may include relevant information for the user to make the decision including the store location, a date and time when the consumable product may be ready for pickup. The user may subsequently select the store and pickup date desired from the presented options.

Alternatively, the user may pre-select a store location or request a favored store location where the pickup may be placed with. The user may save the preference to the user profile or account linked to the sensor device in step 709. Based on the user's location, date and time selection, the reorder management system 123 may transmit the pickup request and profile information to the selected store in order to arrange the pickup as agreed upon by the user.

In some embodiments of the method, the present action set in step 703 may trigger the action of adding the consumable product to a virtual list in step 721. The virtual list may be maintained by the computer system 101, a network accessible computer system such as reorder management system 123 or a remotely accessible data store 125. Upon triggering the pre-set action in step 721, the computer system may load and/or edit a virtual list in the memory device 116, if the virtual list is stored locally on the computer system 101. Likewise, if the virtual list is stored by a remotely accessible computer system, the system 100 may transmit a service request to retrieve the virtual list from the network resource and then upon receiving the virtual list data, load the virtual list in the memory device 116 of the computer system. The system 100 may edit the virtual list by adding the consumable product to the list automatically for the user. Thus, when the user opens the virtual list in the computer system 101 (or any other computer system having the list application), the virtual list may display on the display device 130, the consumable product along with any other items that may have been added automatically by the system 100 or that the user may have manually added.

Alternatively, in an embodiment of the method 700 wherein the pre-set action of step 703 is selected to display an alert to the user when the consumable product has depleted below the selected level, the computer system 101, upon receiving sensor data from the sensor module 109 may instruct the reporting module 115 to display the alert on the computer system 101. For example, the reporting module 115 may display a text or graphical based notification on the display device 130 connected to the computer system 101 or other output device. The notification or alert displayed by the computer system 101 may be a reminder to reorder the consumable product or simply a notification that the particular consumable product's level has dipped below the pre-set height of the sensor device 201 positioned on the exterior surface of the consumable product container. The user may subsequently manually purchase the replacement consumable product after being notified.

In some embodiments of the method 700, the pre-set action in 703 may be to display an alert or reminder as a function of geolocation. The system may trigger the pre-set action in step 721 however the system 100 may delay the transmission or display of the alert or reminder until the user or computer system 101 is at a specified geolocation. For example, the system 100 may create one or more geofences around nearby stores that may stock the consumable product that is currently depleted below the designated level. The computer 101 may transmit a signal to the reporting module 115 to display the alert or notification to the user regarding the depleted consumable product, when the location module reports to the reporting module 115 location data that may be within the established geofence. The computer system 101 may periodically or continuously track the location data of the location module 107 with the nearest GPS location information of local stores. When the computer system 101 is within a particular distance from the local store (determined as a function of comparing the location information) the reporting module 115 may transmit or display the alert or notification to the user.

In alternative embodiments of method 700, the user may select the action in step 703 to queue the product for pickup, at a local store, as a function of geolocation. The pre-set action to queue the product for pickup may operate in a manner that may be a combination of the pickup action and the geolocation based alert. Once the method has triggered the pre-set action in step 721, the computer system 101 may compare the location data received from the location module 107 with location information of local stores stocking the consumable product, which has been depleted. When the location data of computer system 101 is either within a specific distance of the local store, or the computer system 101 has crossed the boundary of a geofence surrounding the store, the computer system 101 may schedule a pickup of the consumable product at the local store by transmitting a service request to the reorder management system 123. The reorder management system forwards the request to the local store and arranges for the pickup to be performed by the user upon arrive or with a specified time period of arrival.

In some embodiments, upon meeting the location requirements, the computer system service request to the reorder management system 123 to handle the pickup transaction. The service request to the reorder management system 123 may include identifying information about one or more consumable products that are being ordered, profile and/or account information of the user as well as location information of the user. Upon receiving the service request, the reorder management system 123 may estimate the time to schedule the pickup of the consumable product(s) and further request the local store to prepare and collect the items for pickup by the account holder at the estimated time. The user, upon entering the nearby local store, may proceed to pick up the consumable product(s) without personally having to collect the products from the local store's shelves. Instead, the consumable product may be pre-arranged and ready for checkout. The computer system 101 may allow for the user to utilize the user's account linked to the reorder management system to pre-pay for the consumable products via the reordering module 103, or the user may arrange for payment to be processed at the local store upon pickup.

After fulfilling the performance of the pre-set action in step 721, the method may proceed to step 722, wherein the user has received a replacement consumable product as a result of the pre-set action, either directly through arranging a pick up or delivery or indirectly using reminders or virtual lists to encourage the user to replace the depleted consumable product. The user may proceed to revert back to step 713 by affixing the sensor device 201 to the exterior of the replacement consumable device as described in step 713 above.

### Computer System

Referring to the drawings, FIG. 8 illustrates a block diagram of a computer system 800 that may be included in the systems of FIGS. 1-6 and for implementing methods for reordering a consumable product as shown in the embodiment of FIG. 7 and in accordance with the embodiments of the present disclosure. The computer system 800 may generally comprise a processor, otherwise referred to as a central processing unit (CPU) 891, an input device 892 coupled to the processor 891, an output device 893 coupled to the processor 891, and memory devices 894 and 895 each coupled to the processor 891. The input device 892, output device 893 and memory devices 894, 895 may each be coupled to the processor 891 via a bus. Processor 891 may perform computations and control the functions of computer 800, including executing instructions included in the computer code 897 for tools and programs for reordering a consumable product, in the manner prescribed by the embodiments of the disclosure using the systems of FIGS. 1-6, wherein the instructions of the computer code 897 may be executed by processor 891 via memory device 895. The computer code 897 may include software or program instructions that may implement one or more algorithms for reordering a consumable product, as described in detail above. The processor 891 executes the computer code 897. Processor 891 may include a single processing unit, or may be distributed across one or more processing units in one or more locations (e.g., on a client and server).

The memory device 894 may include input data 896. The input data 896 includes any inputs required by the computer code 897. The output device 893 displays output from the computer code 897. Either or both memory devices 894 and 895 may be used as a computer usable storage medium (or program storage device) having a computer readable program embodied therein and/or having other data stored therein, wherein the computer readable program comprises the computer code 897. Generally, a computer program product (or, alternatively, an article of manufacture) of the computer system 800 may comprise said computer usable storage medium (or said program storage device).

Memory devices 894, 895 include any known computer readable storage medium, including those described in detail below. In one embodiment, cache memory elements of memory devices 894, 895 may provide temporary storage of at least some program code (e.g., computer code 897) in order to reduce the number of times code must be retrieved from bulk storage while instructions of the computer code 897 are executed. Moreover, similar to processor 891, memory devices 894, 895 may reside at a single physical location, including one or more types of data storage, or be distributed across a plurality of physical systems in various forms. Further, memory devices 894, 895 can include data distributed across, for example, a local area network (LAN) or a wide area network (WAN). Further, memory devices 894, 895 may include an operating system (not shown) and may include other systems not shown in FIGS. 1-6.

In some embodiments, the computer system 800 may further be coupled to an Input/output (I/O) interface and a computer data storage unit. An I/O interface may include any system for exchanging information to or from an input device 892 or output device 893. The input device 892 may be, inter alia, a keyboard, a mouse, sensors, biometric input device, camera, etc. The output device 893 may be, inter alia, a printer, a plotter, a display device (such as a computer screen or monitor), a magnetic tape, a removable hard disk, a floppy disk, etc. The memory devices 894 and 895 may be, inter alia, a hard disk, a floppy disk, a magnetic tape, an optical storage such as a compact disc (CD) or a digital video disc (DVD), a dynamic random access memory (DRAM), a read-only memory (ROM), etc. The bus may provide a communication link between each of the components in the computer system 800, and may include any type of transmission link, including electrical, optical, wireless, etc.

An I/O interface may allow computer system 800 to store information (e.g., data or program instructions such as program code 897) on and retrieve the information from computer data storage unit (not shown). Computer data storage unit includes a known computer-readable storage medium, which is described below. In one embodiment, computer data storage unit may be a non-volatile data storage device, such as a magnetic disk drive (i.e., hard disk drive) or an optical disc drive (e.g., a CD-ROM drive which receives a CD-ROM disk).

As will be appreciated by one skilled in the art, in a first embodiment, the present invention may be a method; in a second embodiment, the present invention may be a system; and in a third embodiment, the present invention may be a computer program product. Any of the components of the embodiments of the present invention can be deployed, managed, serviced, etc. by a service provider that offers to deploy or integrate computing infrastructure with respect to reordering a consumable product. Thus, an embodiment of the present disclosure discloses a process for supporting computer infrastructure, where the process includes providing at least one support service for at least one of integrating, hosting, maintaining and deploying computer-readable code (e.g., program code 897) in a computer system (e.g., computer 800) including one or more processor(s) 891, wherein the processor(s) carry out instructions contained in the computer code 897 causing the computer system to reorder a consumable product. Another embodiment discloses a process for supporting computer infrastructure, where the process includes integrating computer-readable program code into a computer system including a processor.

The step of integrating includes storing the program code in a computer-readable storage device of the computer system through use of the processor. The program code, upon being executed by the processor, implements a method of reordering a consumable product. Thus the present invention discloses a process for supporting, deploying and/or integrating computer infrastructure, integrating, hosting, maintaining, and deploying computer-readable code into the computer system 800, wherein the code in combination with the computer system 800 is capable of performing a method for reordering a consumable product.

A computer program product of the present invention comprises one or more computer readable hardware storage devices having computer readable program code stored therein, said program code containing instructions executable by one or more processors of a computer system to implement the methods of the present invention.

A computer program product of the present invention comprises one or more computer readable hardware storage devices having computer readable program code stored therein, said program code containing instructions executable by one or more processors of a computer system to implement the methods of the present invention.

A computer system of the present invention comprises one or more processors, one or more memories, and one or more computer readable hardware storage devices, said one or more hardware storage devices containing program code executable by the one or more processors via the one or more memories to implement the methods of the present invention.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed:

1. A method for reordering a consumable product comprising the steps of:
affixing a sensor device comprising a sensor and an adjustable band to an exterior surface of a container confining the consumable product, wherein a height of the sensor device affixed to the container is at a desired level of the consumable product remaining before triggering a pre-set action;
calculating, by the sensor device, a density measurement of the consumable product inside the container;
detecting, by the sensor device, a change in the density measurement, indicating depletion of the consumable product below the desired level; and
transmitting, by the sensor device, a signal to perform the pre-set action as a function of the detecting step.

2. The method of claim 1, wherein the pre-set action is selected from the group consisting of scheduling a delivery of the consumable product, scheduling a pickup of the consumable product, queuing an order of the consumable product for pickup at a store upon entering a specified geolocation of the store and displaying a reminder notification upon entering the specified geolocation.

3. The method of claim 1, wherein the sensor of the sensor device is selected from the group consisting of a capacitive sensor, a light sensor and a radar sensor.

4. The method of claim 1, wherein the step of detecting the change in the density measurement is performed using a dielectric constant to determine, by the sensor device, a capacitance differential generated as a function of the density measurement in a presence of the consumable product inside the container and in an absence of the consumable product inside the container.

5. The method of claim 1, further comprising the step of:
electronically pairing the sensor device with a computer system;
electronically linking the sensor device to a user account loaded into a computer readable storage device of a computer system; and
receiving, by the computer system, the signal to perform the pre-set action as a function of the detecting step.

6. The method of claim 5, further comprising the step of:
performing, by the computer system, the pre-set action as a function of a geolocation of the computer system.

7. The method of claim 1, further comprising the step of:
identifying, by the sensor device, the consumable product, wherein the step of identifying is performed by scanning the consumable product or scanning a barcode attached to the consumable product.

8. A computer system, comprising:
a central processing unit (CPU);
a memory device coupled to the CPU;
a sensor device, comprising a sensor and an adjustable band, the sensor device coupled to the CPU; and
a computer readable storage device coupled to the CPU, wherein the computer readable storage device comprises program code executable by the CPU via the memory device to implement a method for reordering a consumable product comprising the steps of:
calculating, by the sensor device, a density measurement of the consumable product stored inside a container;
detecting, by the sensor device, a change in the density measurement, indicating a removal of the consumable product below a desired level, wherein the desired level is indicated by a position of the sensor device affixed to an exterior surface of the container; and
transmitting, by the sensor device, a signal to perform a pre-set action by the CPU, as a function of the detecting step.

9. The system of claim 8, wherein the pre-set action is selected from the group consisting of scheduling a delivery of the consumable product, scheduling a pickup of the consumable product, queuing an order of the consumable product for pickup at a store upon entering a specified geolocation of the store and displaying a reminder notification upon entering the specified geolocation.

10. The system of claim 8, wherein the sensor of the sensor device is selected from the group consisting of a capacitive sensor, a light sensor and a radar sensor.

11. The system of claim 8, wherein the step of detecting the change in the density measurement is performed using a dielectric constant to determine, by the sensor device, a capacitance differential generated as a function of the density measurement in a presence and an absence of the consumable product inside the container.

12. The system of claim 8, further comprising:
accessing, by the CPU, a user account electronically linked to the sensor device, wherein the user account is loaded into the memory device;
receiving, by the CPU, the signal to perform the pre-set action; and
performing, by the CPU, the pre-set action.

13. The system of claim 12, wherein performing the pre-set action by the CPU occurs as a function of a geolocation.

14. The system of claim 8, wherein the computer system configures the sensor device, to identify a consumable product being detected by scanning the consumable product or scanning a tag identifying the consumable product.

15. A computer program product comprising:
a computer-readable storage device; and
a computer-readable program code stored in the computer-readable storage device, the computer-readable program code comprising instructions executed by a central processing unit (CPU) of a computer system implementing a method for reordering a consumable product comprising the steps of:
calculating, by the CPU, a density measurement of the consumable product stored inside a container from data collected by a sensor device electronically paired to the computer system;
detecting, by the CPU, a change in the density measurement from the data collected by the sensor device, indicating a removal of the consumable product below a desired level, wherein the desired level is indicated by a position of the sensor device affixed to an exterior surface of the container;
receiving, by the CPU, a signal to perform the pre-set action from the sensor device; and
performing, by the CPU, the pre-set action as a function of the detecting step.

16. The computer program product of claim 15, wherein the pre-set action is selected from the group consisting of scheduling a delivery of the consumable product, scheduling a pickup of the consumable product, queuing an order of the consumable product for pickup at a store upon entering a specified geolocation of the store and displaying a reminder notification upon entering the specified geolocation.

17. The computer program product of claim 15, wherein the sensor device comprises a sensor selected from the group consisting of a capacitive sensor, a light sensor and a radar sensor.

18. The computer program product of claim 15, wherein the step of detecting the change in the density measurement is performed using a dielectric constant to determine, by the sensor device, a capacitance differential generated as a function of the density measurement in a presence and an absence of the consumable product inside the container.

19. The computer program product of claim 15 further comprising the steps of:
accessing, by the CPU, a user account electronically linked to the sensor device; and
recording, by the CPU, fulfillment of the pre-set action to the user account.

20. The computer program product of claim 15, wherein the step of performing the pre-set action by the CPU occurs as a function of a geolocation.
